(19) United States
(12) Patent Application Publication
(10) Pub. No.: US 2006/0100680 A1
(43) Pub. Date: May 11, 2006

(54) Bath blanket

(76) Inventor: Joan M. Brikman, Brooklyn, NY (US)

Correspondence Address: LITMAN LAW OFFICES, LTD, PO BOX 15035, CRYSTAL CITY STATION, ARLINGTON, VA, 22215, US

(21) Appl. No.: 11/253,649

(22) Filed: Oct. 20, 2005

Related U.S. Application Data
(60) Provisional application No. 60/620,772, filed on Oct. 21, 2004.

(51) Int. Cl. A61F 7/00

(52) U.S. Cl. 607/96

(57) ABSTRACT

The bath blanket has a heat-absorbing inner gel layer, a thin plastic gel retaining liner completely surrounding the gel layer, and an external cloth layer attached or adhered to at least one side of the retaining liner. The gel layer may absorb heat by absorbing microwave energy in a microwave oven, and is non-toxic. The external cloth layer is preferably a lightweight, thin terry cloth fabric.

### CROSS-REFERENCE TO RELATED APPLICATION

This application claims the benefit of U.S. Provisional Patent Application Ser. No. 60/620,772, filed Oct. 21, 2004.

### BACKGROUND OF THE INVENTION

1. Field of the Invention

The present invention relates to blankets and heatable covers, and particularly to a gel filled blanket that can be preheated in a microwave and used to cover portions of the body not submerged in the bath tub to keep the person's body warm.

2. Description of the Related Art

When bathing, many people prefer to sit or recline in the bathtub to relax in warm water. However, conventional bathtubs are typically too short for an adult to immerse his or her body in the warm water. Of course, the head must remain above water. But if the feet are submerged, the neck and shoulders are exposed, and vice versa, unless the person sits with his or her knees flexed above water. Sometimes both the shoulders and the flexed knees are exposed. If there's a draft in the room, the exposed portion of the body is chilled, which detracts from the comfort and relaxation the bather seeks.

One can try to drape the exposed portion of the body with a towel or blanket. However, while providing a shield from direct exposure to the draft, such makeshift measures still leave the body chilled. While hot packs are available that can be preheated in a microwave or a pan of hot water, such hot packs must be wrapped in a towel, sheet, or other wrapper, and are difficult to drape over the exposed portion of the body when bathing. Electric blankets or heating pads are often oversized, and their use when bathing poses safety hazards.

Consequently, there is a need for a warm, waterproof cover for the part of the body that remains unsubmerged while a person is in the bathtub. Thus, a bath blanket solving the aforementioned problems is desired.

### SUMMARY OF THE INVENTION

The bath blanket has a heat-absorbing inner gel layer, a thin plastic gel retaining liner completely surrounding the gel layer, and an external cloth layer attached or adhered to at least one side of the retaining liner. The gel layer may absorb heat by absorbing microwave energy in a microwave oven, and is non-toxic. The external cloth layer is preferably a lightweight, thin terry cloth fabric.

The bath blanket is waterproof and is about one-eighth of an inch in total thickness. Additionally, the length and width of the blanket may be sized to accommodate use of the blanket across a user in a bathtub.

The present invention is also directed towards a method of using a microwavable gel-filled blanket having a lightweight, thin terry cloth fabric on an exterior portion of the blanket, and includes placing the gel-filled blanket in a microwave and operating the microwave for a period of time to heat the gel filled portion of the blanket to a desired temperature.

The heated, gel-filled blanket is removed from the microwave and the fabric portion of the blanket is placed on a user to transmit heat from the gel-filled blanket onto the parts of the user's body in contact with the fabric portion.

These and other features of the present invention will become readily apparent upon further review of the following specification and drawings.

### BRIEF DESCRIPTION OF THE DRAWINGS

FIG. 1 is an environmental, perspective view of a bath blanket according to the present invention in use by a person in a bathtub.

FIG. 2 is a fragmented perspective view of the bath blanket of the present invention, broken away and partly in section to show details of the construction of the blanket.

Similar reference characters denote corresponding features consistently throughout the attached drawings.

### DETAILED DESCRIPTION

The bath blanket of the present invention provides a warm, waterproof cover for the part of the body that remains unsubmerged while a person is in the bathtub. The blanket would likely be utilized by people who have a standard-size bathtub, in which they do not have room to stretch out and submerge their entire body.

The blanket is designed to be microwavable, so that it would already be warm or hot when it is placed on the body.

The blanket may be made of a microwavable plastic, containing a non-toxic heat retaining gel. It would be covered with a lightweight, thin layer of terry cloth on one side, so that the user could place this side against the body in order to feel more comfortable. The cloth side is intended to feel like a washcloth, but would conduct heat from the heated gel layer for warmth.

The possibility exists for manufacturing the blanket in various sizes to accommodate thinner or full figure or portly body types.

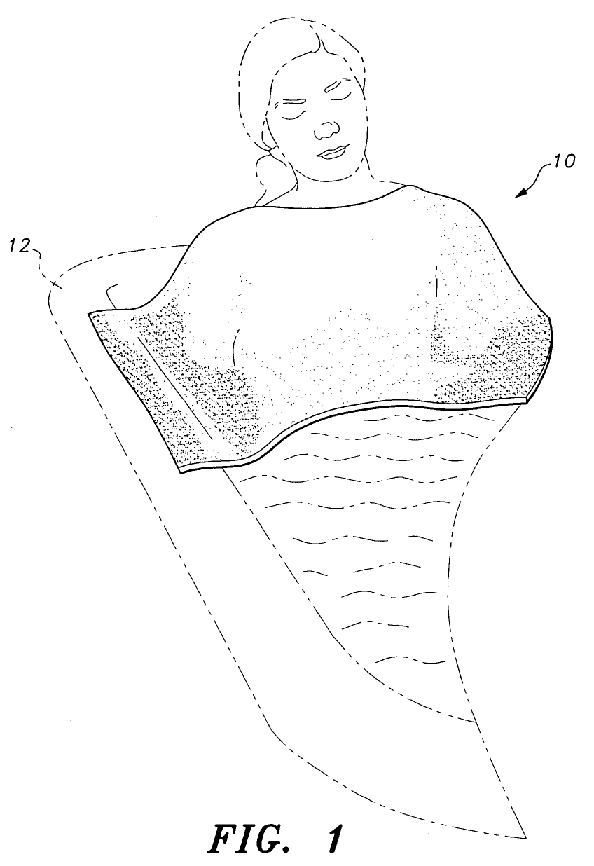

FIG. 1 shows an environmental view of the bath blanket 10 draped over portions of the body of a user reclining in a bathtub 12. The blanket 10 may be draped across an unsubmerged portion of the user and across the bathtub. The blanket 10 may also be of a smaller size to only drape across the user and not the edges of the bathtub 12.

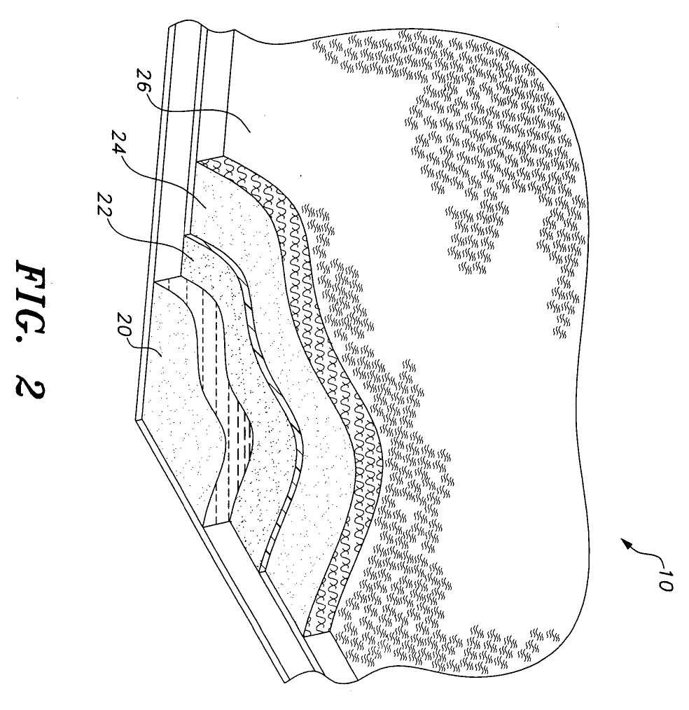

FIG. 2 shows details of the structure of the blanket 10. A thin microwavable plastic cover 20 and 24 forms a pack or envelope that fully contains a non-toxic heating gel 22 therein. The thin microwavable plastic cover 20 and 24 presents a waterproof barrier when any portion of the blanket 10 is submerged in the bathtub 12. The gel pack or envelope is preferably thin (about ⅛″ thick) and relatively flexible so that the blanket is foldable for placement in a microwave.

A thin, soft cloth covering 26 is attached or adhered to an outer surface of the thin microwavable plastic cover 24. The thin, soft cloth covering 26 is preferably made from lightweight terry cloth, or any other soft fabric material that would be soft to the touch of the user in a bathtub environment.

The blanket 10 may be manufactured in any size necessary to accommodate any sized person in a bathtub environment.

It is to be understood that the present invention is not limited to the embodiments described above, but encompasses any and all embodiments within the scope of the following claims.

I claim:

1. A blanket, comprising:
a thermal gel pack having a plastic cover and thin layer of gel enclosed within the plastic cover, the gel pack being elongated and flat and having a first surface and a second surface, the gel pack being heatable in a microwave oven; and
a layer of soft, lightweight cloth attached to the first surface of the gel pack in order to form a heatable blanket.

2. The blanket of claim 1, wherein said gel layer is made from non-toxic material.

3. The blanket of claim and 1, wherein said gel pack absorbs microwave energy, whereby said gel pack is capable of being heated in a microwave oven.

4. The blanket of claim 1, said cloth layer comprises a lightweight, thin terry cloth fabric.

5. The blanket of claim 1, wherein said heatable blanket is waterproof.

6. The blanket of claim 1, wherein said gel-pack is about one-eighth of an inch thick.

7. The blanket of claim 1, wherein said layer of cloth and the first surface of said gel pack are co-extensive in size.

8. The blanket according to claim 1, wherein said heatable blanket is dimensioned and configured for draping over an exposed portion of a person's body when bathing in a bathtub in order to keep the exposed portion warm.

9. A method of using a microwavable gel-filled blanket having a lightweight, thin terry cloth fabric on an exterior portion of said blanket, the method comprising the steps of:
placing said gel-filled blanket in a microwave;
operating said microwave for a period of time to heat the gel filled portion of said blanket to a desired temperature;
removing said heated gel-filled blanket from said microwave; and
placing said fabric portion of said heated gel-filled blanket on a user to transmit heat from said gel-filled blanket to the exposed portions of the user's body.
